(19) United States
(12) Patent Application Publication
(10) Pub. No.: US 2012/0022391 A1
(43) Pub. Date: Jan. 26, 2012

(54) Multimodal Brain Computer Interface

(75) Inventor: Eric C. Leuthardt, St. Louis, MO (US)

(73) Assignee: WASHINGTON UNIVERSITY IN ST. LOUIS, St. Louis, MO (US)

(21) Appl. No.: 13/188,965

(22) Filed: Jul. 22, 2011

Related U.S. Application Data
(60) Provisional application No. 61/366,656, filed on Jul. 22, 2010.

(51) Int. Cl. A61B 5/0476

(52) U.S. Cl. 600/544

(57) ABSTRACT

Determining an intended action based on one more cortico-physiologies within brain signals includes establishing communication with one or more electrodes for sensing the brain signals of a subject, and concurrently receiving brain signals representative of a plurality of cortico-physiologies. The brain signals are transmitted to a processor for use in determining the intended action and controlling a device.

### CROSS REFERENCE TO RELATED APPLICATIONS

This application claims the benefit of Provisional Patent Application Ser. No. 61/366,656, entitled “MULTIMODAL BRAIN COMPUTER INTERFACE”, which was filed on Jul. 22, 2010 and which is hereby incorporated by reference in its entirety.

### BACKGROUND

Embodiments described herein relate generally to brain computer interfaces and, more particularly, to using a plurality of cognitive processes and their associated physiologic signatures concurrently, to achieve brain computer device control.

At least some known brain computer interfaces (BCIs) use a single cognitive operation and its associated cortical physiology as a signal for controlling a machine. For example, classically, the single cognitive process and its associated physiology used for device control has been cortical signals, such as neuron firing, EEG, or ECoG signals. However, such known BCIs provide little benefit to motor-impaired subjects that have suffered a unilateral stroke, for example, because such known BCIs rely on brain signals that originate from the contralateral motor cortex, which is generally the same region injured by the stroke.

### BRIEF DESCRIPTION OF THE DRAWINGS

The embodiments described herein may be better understood by referring to the following description in conjunction with the accompanying drawings.

FIG. 1 is a block diagram of an exemplary brain computer interface (BCI).

FIG. 2 is a block diagram of signal acquisition circuitry that may be used with the BCI shown in FIG. 1.

FIG. 3 is a block diagram of signal analysis circuitry that may be used with the BCI shown in FIG. 1.

FIG. 4 is a flowchart that illustrates an exemplary method for controlling a device based on one more cognitive processes using the BCI shown in FIG. 1.

FIGS. 5A, 5B, and 5C are graphs that illustrate ipsilateral hand movements that produce changes in lower frequencies and in different cortical sites, and that occur earlier than contralateral hand movements.

FIG. 6 is a performance curve that illustrates using signals associated with ipsilateral movements for controlling an external device.

FIGS. 7A and 7B illustrate that motor and speech intentions are separable by anatomic location and frequency content associated with cortical activation.

FIGS. 8A and 8B illustrate that phonemic classes of words are separable by cortical networks.

FIG. 9 is a performance curve that illustrates utilization of real and imagined phoneme articulation for target selection.

FIG. 10 is a graph that illustrates rapid changes induced in ECoG-BCI control signals in humans using biofeedback.

FIGS. 11A and 11B illustrate inducing separable control signals using biofeedback.

### DETAILED DESCRIPTION

The embodiments described herein enable a brain computer interface to concurrently use cortical physiologies associated with different cognitive processes for device control. Beyond combining ipsilateral motor signals and contralateral motor signals, the embodiments described herein combine other modalities to provide, for example, the combination of speech signals with motor signals. Other signals that may also be selectively combined include attention-related signals, signals related to cortical plasticity or feedback, signals related to working memory, signals related to higher cognitive operations (e.g. mathematical processing), signals related to auditory processing, and/or signals related to visual perception.

Moreover, the embodiments described herein enable integration of motor and non-motor cortical physiologies for brain computer interface control. By better defining the cortical signals associated with human motor function, speech, and cortical plasticity and their utility in augmenting current neuroprosthetic operation, the embodiments described herein enable the potential for enhanced control capabilities of electrocorticographic derived brain computer interfaces.

To facilitate understanding of the embodiments described herein, certain terms are defined below.

In some embodiments, the term “electrocorticography” and the acronym “ECoG” refer generally to a technique that involves recording surface cortical potentials from either epidural or subdural electrodes.

In some embodiments, the term “brain computer interface” and the acronym “BCI” refer generally to signal-processing circuitry that acquires input in the form of raw cortical brain signals and converts the brain signals to a processed signal that is output to a computer for storage and/or further analysis. Moreover, in some embodiments, the term “BCI system” refers generally to a number of components, including a BCI, that translates raw brain signals into control of a device.

In some embodiments, the term “device” refers generally to equipment or a mechanism that is designed to provide a special purpose or function. Exemplary devices including, but are not limited to, a cursor on a video monitor, computer software, environmental controls, entertainment devices, implantable controls, prosthetics, beds, and mobility devices such as wheelchairs or scooters. Moreover, the term also includes input devices that are used to control other devices such as those that are listed above. Exemplary input devices include, but are not limited to, wheels, joysticks, levers, buttons, keyboard keys, trackpads, and trackballs.

In some embodiments, the cortical signals are ECoG signals. However, the cortical signals may also be one or more of electroencephalography (EEG) signals, local field potentials, single neuron signals, magnetoencephalography (MEG) signals, multi-unit, or mu rhythm signals, beta rhythm signals, low gamma rhythm signals, high gamma rhythm signals, and the like. Moreover, the ECoG signals, EEG signals, local field potentials, and/or MEG signals may include one or more of mu rhythm signals, beta rhythm signals, low gamma rhythm signals, and high gamma rhythm signals. The signal data is converted into the frequency domain and cortical physiologies associated with different cognitive processes are identified with regards to frequency, phase, phase-amplitude coupling, location, and timing. The embodiments described herein enables high signal resolution associated with ECoG, for example, to reveal aspects of cortical signal processing that is unavailable with noninvasive means.

A substantial percentage of stroke sufferers are left with a permanent hemiparesis, which typically includes an acute decrement in hand function. However, such conditions frequently show some recovery over time by using the undamaged brain hemisphere that is ipsilateral to the affected limb. Portions of the brain associated with ipsilateral motor movements are anatomically and temporally distinct from the locations and timing associated with contralateral limb movements. However, there are physiologic features that distinguish not only cortical processing for ipsilateral and contralateral movement, but also primary motor signals and non-primary motor signals, as well as motor signals and non-motor signals.

The highest level of functioning demonstrated by known BCIs is two-dimensional (2D) control using sensorimotor rhythms recorded from primary motor cortex (M1). More complex control, however, is needed to justify the risks of surgical implantation for clinical trials. Thus, the identification of additional control features enables patients to navigate routine computer software as if they were using a hand-held mouse (e.g., 2-D control plus a “click” function), or to have the ability to manipulate an object in the environment (three-dimensional control). This baseline level of functional improvement is needed for further translational advancement. As a strategy to improve ECoG-BCI's functional potential, the embodiments described herein identify cognitive processes and cortical physiologies that are distinct from M1 sensorimotor rhythms. Additionally, the embodiments described herein demonstrate that cortical signals outside of M1 can complement and augment current levels of sensorimotor rhythm device control.

For example, non-primary motor physiologies, such as ipsilateral movements, produce electrocorticographic changes that have distinct cortical locations, different temporal onset timings, and/or different frequency spectral alterations when compared against primary motor physiologies, such as contralateral movements. Similarly, non-motor physiologies, such as speech, attention, cortical plasticity and feedback, and auditory processing, produce electrocorticographic changes that have distinct cortical locations, different temporal onset timings, and/or different frequency spectral alterations when compared against motor physiologies, such as ipsilateral or contralateral movements. The unique spatial and spectral electrophysiologic features associated with non-motor physiologies, non-primary motor physiologies, and primary motor physiologies may be effectively used as described herein to control a device.

ECoG-based BCI systems have emerged as a signal platform for neuroprosthetic application. To date, the highest level of machine control has been two-dimensional cursor movements derived from sensorimotor rhythms from primary motor cortex (M1). This experimental demonstration, however, is insufficient to justify the risks of surgical implantation of ECoG-based BCI platforms in patients with spinal cord injury (SCI) or similarly impairments. The current need for higher levels of control prohibits the potential for further translational advancement. Embodiments described herein provide a clinically relevant functionality to patients with severe motor disability due to spinal cord dysfunction. For example, additional control features may be identified using cortical signals outside of M1 that can augment current sensorimotor rhythm BCI operation, which enables ECoG-derived control to be improved by using additional cognitive and cortical processes in parallel with sensorimotor rhythms from M1. Moreover, the embodiments described herein enable identification of separable cortical physiology associated with ipsilateral motor movements to supplement sensorimotor rhythm-derived BCI control, identification of the separable cortical physiology associated with speech to supplement sensorimotor rhythm-derived BCI control, and define the role of biofeedback alone to enable creation of separable features to supplement sensorimotor rhythm-derived BCI control. Specific motor, language, and biofeedback-related tasks resulting in ECoG data may be converted into the frequency domain, and spectral changes may be identified with regard to frequency, location, and timing. Features specific to the various tasks may then be used for various control scenarios to augment standard control derived from M1-cortical signals. In sum, the embodiments described herein provide an understanding of human cortical physiology and provide BCI control signals for the translational development of ECoG-BCIs for patients with SCI and other disabilities.

The embodiments described herein facilitate identifying separable cortical physiology associated with ipsilateral motor movements to supplement sensorimotor rhythm-derived BCI control. Ipsilateral hand movement and movement imagery produce spectral changes located predominantly in premotor cortex and are associated with lower frequencies when compared to M1-sensorimotor rhythms associated with contralateral movements. These unique anatomic/spectral features provide an additional continuous control signal that can be used in parallel with control from M1-derived signals.

Moreover, the embodiments described herein facilitate identifying separable cortical physiology associated with speech to supplement sensorimotor rhythm-derived BCI control. Articulation of different phonemes produce distinct spectral changes in Broca's and Wernicke's areas. These unique anatomic/spectral features provide additional discrete control signals that can be used in parallel with continuous control from M1-derived signals as a discrete selector (e.g., a computer mouse “click” function).

Furthermore, the embodiments described herein facilitate defining the role of biofeedback alone to enable creation of separable features to supplement sensorimotor rhythm-derived BCI control. With appropriate feedback, subjects can alter the amplitude of designated frequencies at designated locations to create “de novo” control features to provide additional discrete control signals for use in parallel with continuous control from M1-derived signals as a discrete selector (e.g., a computer mouse “click” function).

Embodiments of the disclosure contemplate additional control features to augment sensorimotor-derived control and produce numerous cognitive and control strategies to move ECoG-BCI systems closer to a threshold of functional improvement where clinical validation is warranted.

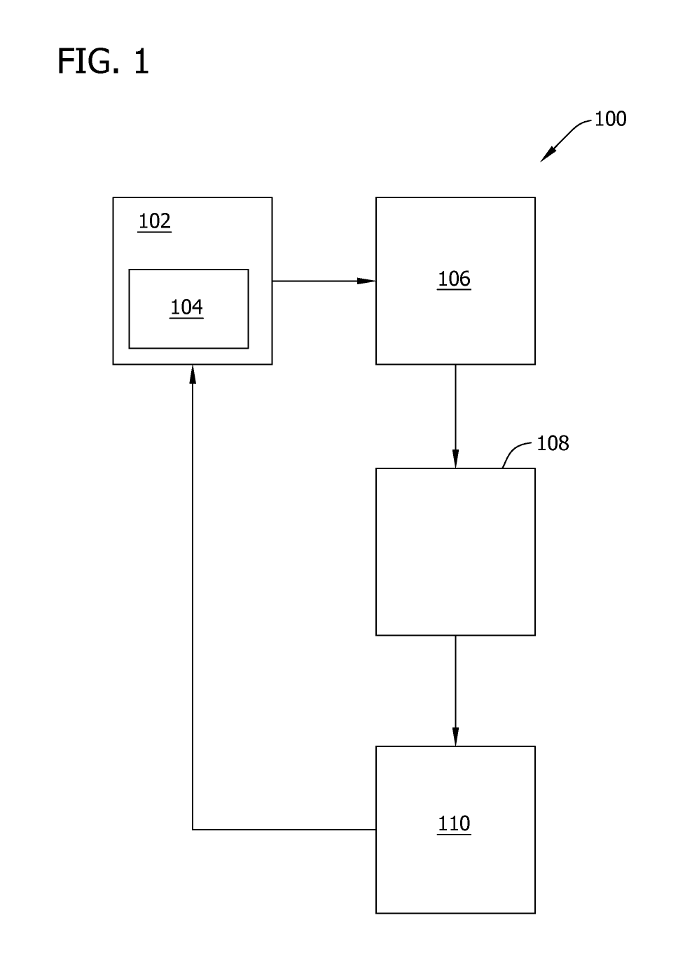

FIG. 1 is a block diagram of an exemplary brain computer interface (BCI) 100 for use acquiring brain signals from a subject's brain 102, translating the brain signals into a control signal, and determining and/or performing an intended action associated with the brain signals. In some embodiments, BCI 100 includes an implantable electrode array 104 that may be positioned either under the dura mater (subdural) or over the dura mater (epidural). In the example of FIG. 1, electrode array 104 is subdural. Electrode array 104 includes a plurality of electrodes (not shown in FIG. 1), such as ECoG electrodes that acquire brain signals from a surface of the brain and generate a raw ECoG signal. Electrode array 104 may be arranged in an 8×8 or 6×8 grid, although other grid arrangements are contemplated. The individual electrodes have a diameter of approximately 4 millimeters (mm) and are composed of, for example, platinum iridium discs. The electrodes are spaced approximately 1 centimeter apart and are encapsulated in silastic sheets, such that separate four-electrode strips are implanted facing the skull (away from the cortical surface) for biosignal amplifier ground and reference. Other configurations include very small electrode arrays that are 75 microns in maximal diameter and spaced 1 mm apart.

BCI 100 also includes signal acquisition circuitry 106 that receives the raw signal from electrode array 104. Signal acquisition circuitry 106 includes, for example, a multiplexer, an amplifier, a filter, an analog-to-digital (A/D) converter, a transceiver, and a power supply (none shown in FIG. 1). An exemplary biosignal amplifier records ECoG signals and microphone data at a sampling frequency of 1.2 kilohertz and 24-bit resolution. Moreover, microphone signals use ground and references electrically isolated from the ECoG signals in order to prevent interference. An exemplary filter is a digital band pass filter that operates between approximately 0.1 Hz and 500 Hz. Signal acquisition circuitry 106 receives the raw signal from electrode array 104 and generates a transmission signal for use in determining an intended action by the subject. In one embodiment, signal acquisition circuitry 106 is included with electrode array 104 in a single, fully-implantable housing. In another embodiment, signal acquisition circuitry 106 is remotely located from electrode array 104. In such an embodiment, electrode array 104 transmits the brain signals to signal acquisition circuitry 106 via a wired connection or via wireless communication. Accordingly, in such an embodiment, electrode array 104 includes a transmitter (not shown in FIG. 1) that enables communication between electrode array 104 and signal acquisition circuitry 106.

Moreover, BCI 100 includes signal analysis circuitry 108, such as a computer. Signal analysis circuitry 108 includes, for example, a memory area and a processor (neither shown in FIG. 1). Signal analysis circuitry 108 receives the transmission signal from signal acquisition circuitry 106, decodes the transmission signal to determine an intended action associated with the brain signals, and generates a control signal for use in controlling a device, such as device 110. For example, signal analysis circuitry 108 decodes the transmission signal, extracts features from the transmission signal, applies a translation algorithm to the features to determine an intended action, and generates the control signal for controlling device 110 according to the intended action. In some embodiments, the memory area includes computer-executable program modules or components (not shown in FIG. 1) that include computer-executable components. One exemplary component includes instructions for synchronizing stimuli presentation and ECoG and microphone signal recording. For example, stimulus periods of approximately four seconds are interleaved between consecutive intertrial intervals (ITI), and visual stimuli is displayed for the entire stimulus period on a display (not shown). In addition, auditory stimuli are presented through headphones. In some embodiments, stimuli for both tasks are selected from a list of monosyllabic and/or polysyllabic English language words.

In one embodiment, signal analysis circuitry 108 is included with electrode array 104 and/or signal acquisition circuitry 106 in a single housing. For example, in one embodiment, electrode array 104, signal acquisition circuitry 106, and/or signal analysis circuitry 108 are integrated into a single circuit chip that is wholly implanted either under the dura mater or over the dura mater. In such an embodiment, control of device 110 is enabled directly from the implanted circuit chip. In another embodiment, signal analysis circuitry 108 is remotely located from electrode array 104 and/or signal acquisition circuitry 106, and communicates with signal acquisition circuitry 106 via a wired connection or via wireless transmission. In yet another embodiment, both signal acquisition circuitry 106 and signal analysis circuitry 108 are remotely located from electrode array 104, and signal acquisition circuitry 106 communicates with electrode array 104 via a wired or via wireless transmission.

BCI 100 is configured to use the unique spatial, temporal, and signal advantages of the brain signals to reveal aspects of cortical processing not possible by noninvasive approaches. The results provide an improvement over known BCIs in that they provide neuroprosthetic strategies to remedy or mitigate motor impairment, such as stroke-induced paralysis, and to restore function. In addition, using ECoG in the exemplary embodiment provides a desirable signal-to-noise ratio, millisecond timescales, millimeter spatial resolution, and a broad frequency bandwidth that, in combination, are not available with other techniques.

Specific frequency alterations within brain signals encode specific information about motor and non-motor actions. Moreover, brain signals from the epidural space may be used to control devices. Taken together, brain signals such as ECoG signals include a high level of specific cortical information and enable a user to effectively gain control of a device. Further, cortical electrophysiologic changes associated with non-primary motor movements, such as ipsilateral movements or speech, are distinct and that these changes support independent thought-driven device control. Embodiments described herein extend that paradigm to provide thought-driven device control based on cortical electrophysiologic changes associated with non-motor physiologies and motor physiologies, including both primary motor physiologies and non-primary motor physiologies.

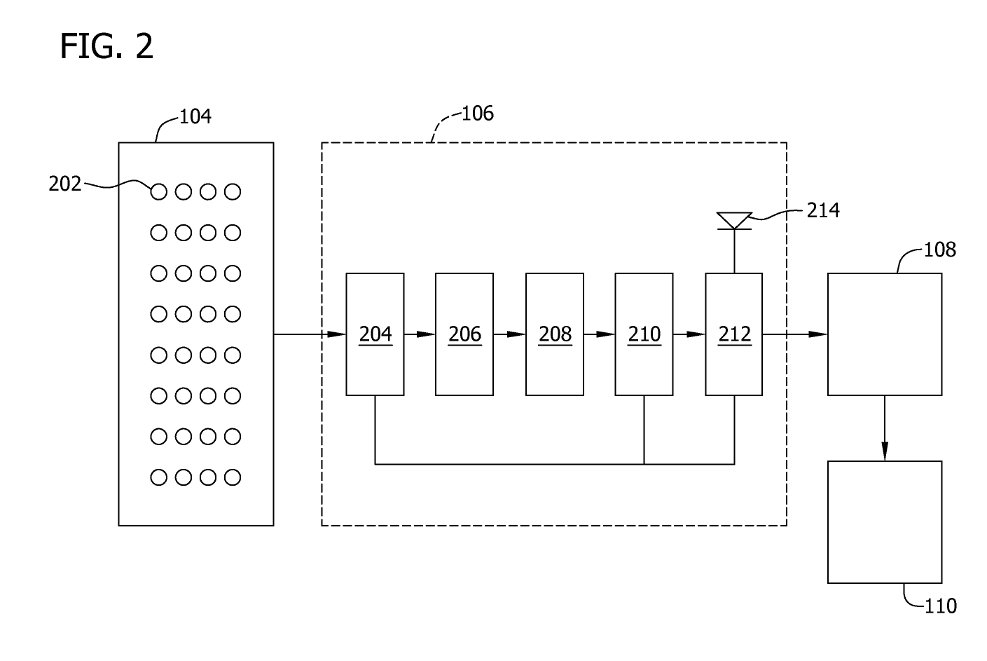

FIG. 2 is a block diagram of signal acquisition circuitry 106. As shown in FIG. 2, signal acquisition circuitry 106 is adapted for communication with electrode array 104 to convert analog brain signals acquired by electrodes 202 to a transmission signal representative of the brain signals. The brain signals are multiplexed, amplified, filtered, and converted from analog to digital. Moreover, in one embodiment, each of the components described below of signal acquisition circuitry 106 are mounted on a flexible substrate, such as a circuit board. Furthermore, in some embodiments, one or more of the components described below are combined such that a single chip provides the functionality described below.

Signal acquisition circuitry 106 includes a multiplexer 204 that receives the brain signals from electrode array 104 via a plurality of channels. For example, in one embodiment, electrode array 104 acquires sixteen channels of analog data. Multiplexer 204 receives the sixteen channels and multiplexes them into a single channel at a desired frequency, such as 8 kHz. In one embodiment, multiplexer 204 switches through each channel and holds the received channel for a selected length of time. Multiplexer 204 holds a signal from a single channel by multiplying the channel by a constant voltage pulse. During a transition time, multiplexer 204 switches to a next channel and adds the multiplied value to the single output channel.

Moreover, signal acquisition circuitry 106 includes an amplifier 206 coupled to multiplexer 204, and a low-pass filter 208 coupled to amplifier 206. Filter 208 removes high-frequency distortions from the amplified signal and prevents aliasing before the signal is converted from analog to digital. An analog-to-digital (A/D) converter 210 synchronizes with multiplexer 204 and with a clock signal supplied by a transmitter 212. In addition, A/D converter 210 addresses each channel within the signal to localize portions of the signal to respective electrodes 202. A/D converter 210 outputs a digital transmission signal to transmitter 212, which is transmitted to signal analysis circuitry 108 via an antenna 214. An exemplary transmitter 212 is a Bluetooth® transmitter (Bluetooth® is a registered trademark of Bluetooth Sig, Inc., Bellevue, Wash., USA). However, any suitable wireless or wired transmitter may be used.

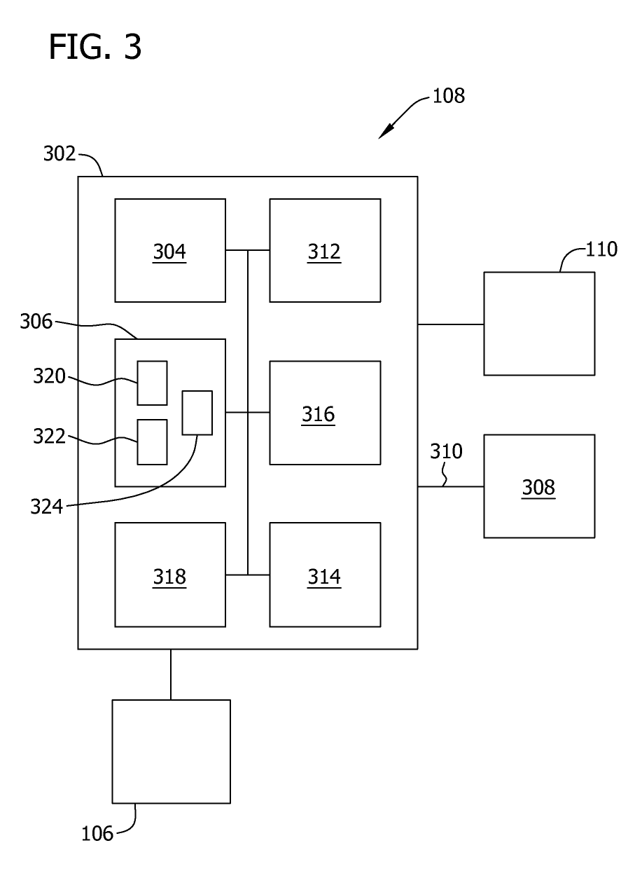

FIG. 3 is a block diagram of signal analysis circuitry 108. In the exemplary embodiment, signal analysis circuitry 108 is embodied as a computer 302. However, any suitable form may be used, such as a Personal Digital Assistant (PDA), a Smartphone, or any other suitably equipped communication device. As shown in FIG. 3, computer 302 includes a processor 304 and a memory area 306 coupled to processor 304. In some embodiments, computer 302 includes multiple processors 304 and/or multiple memory areas 306. Moreover, memory area 306 may be embodied as any suitable memory device or application including, but not limited to, a database, a hard disk device, a solid state device, or any other device suitable for storing data as described herein. Furthermore, in the exemplary embodiment, memory area 306 is located within computer 302. Alternatively, memory area 306 may include any memory area internal to, external to, or accessible by computer 302. Further, memory area 306 or any of the data stored thereon may be associated with any server or other computer, local or remote from computer 302 (e.g., a second computer 308 coupled to computer 302 via a network 310).

Computer 302 includes a display device 312, a secondary storage device 314 such as a writable or re-writable optical disk, and input/output devices 316 such as a keyboard, a mouse, a digitizer, and/or a speech processing unit. In addition, computer 302 includes a transceiver 318 that receives the digital transmission signal from transmitter 212 (shown in FIG. 2) and transmits a control signal to device 110.

In some embodiments, memory area 306 includes one or more computer-readable storage media having computer-executable components. For example, memory area 306 includes a communication component 320 that causes processor 304 to receive the digital transmission signal from signal acquisition circuitry 106 via transceiver 318, a signal analysis component 322 that converts the received signal into a control signal for use in controlling device 110 according to an intended action by the subject, and a control component 324 that uses the control signal to control device 110.

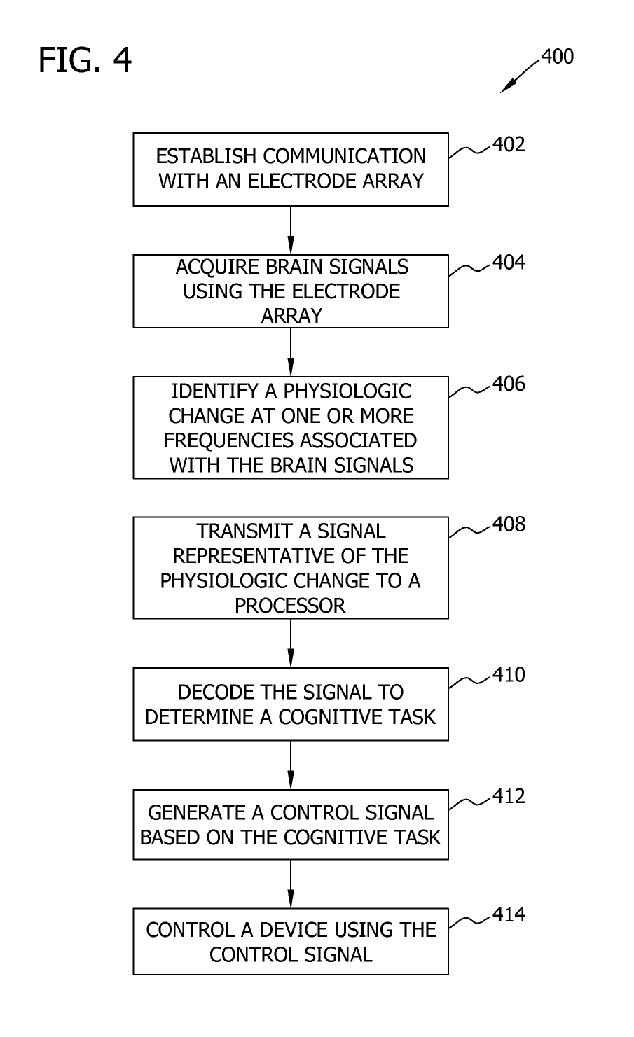

FIG. 4 is a flowchart 400 that illustrates an exemplary method of controlling a device, such as device 110 (shown in FIG. 1) based on physiologies associated with one or more brain signals. Initially, communication is established 402 with electrode array 104 (shown in FIG. 1) implanted beneath the scalp of a subject. Communication may be established via a wired or wireless connection between electrode array 104 and signal acquisition circuitry 106 (shown in FIG. 1). Electrode array 104 acquires brain signals via a plurality of electrodes 202 (shown in FIG. 2) at a single portion of the brain or at multiple portions of the brain simultaneously. Specifically, electrodes 202 concurrently interrogate 404 the brain to obtain multiple cognitive operations and cortico-physiologies. The brain signals acquired by electrodes 202 include motor signals and non-motor signals. Moreover, the motor signals may be primary motor signals and/or non-primary motor signals.

Signal acquisition circuitry 106 receives the brain signals and processes the brain signals to generate a transmission signal, using multiplexer 204, amplifier 206, low-pass filter 208, and analog-to-digital converter 210 (each shown in FIG. 2). Signal acquisition circuitry 106 then transmits 406 the transmission signal to signal analysis circuitry 108 (shown in FIG. 1) via, for example, transmitter 212 (shown in FIG. 2).

Signal analysis circuitry 108 receives the transmission signal via transceiver 318 (shown in FIG. 3), and decodes the transmission signal using processor 304 (shown in FIG. 3). In some embodiments, signal analysis circuitry 108 stores the decoded transmission signal in memory area 306 or in secondary storage 314 (both shown in FIG. 3). Processor 304 determines 408 an intended cognitive task based on stored correlations between the cortico-physiologies and one or more cognitive tasks which are related to intended actions. Signal analysis circuitry 108 generates 410 a control signal representative of the intended action, and controls 412 device 110 using the control signal.

The embodiments described herein focus on understanding how the human cortex encodes intentions and on the development of methods to utilize this information to create BCIs that facilitate function for people with severe motor disabilities. Moreover, the embodiments described herein facilitate identifying additional control features using cortical signals outside of M1 that can augment current sensorimotor rhythm BCI operation. ECoG-derived control is improved by using additional cognitive and cortical processes in parallel with sensorimotor rhythms from M1.

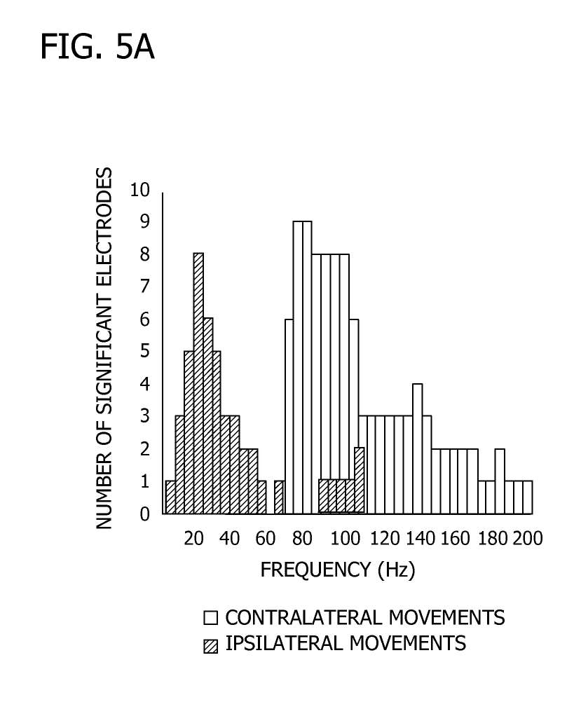

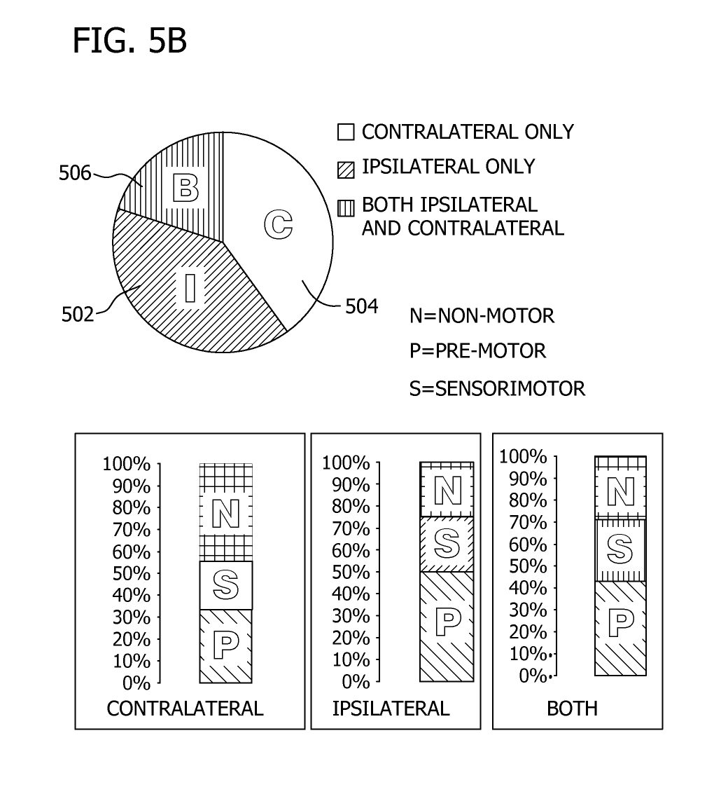

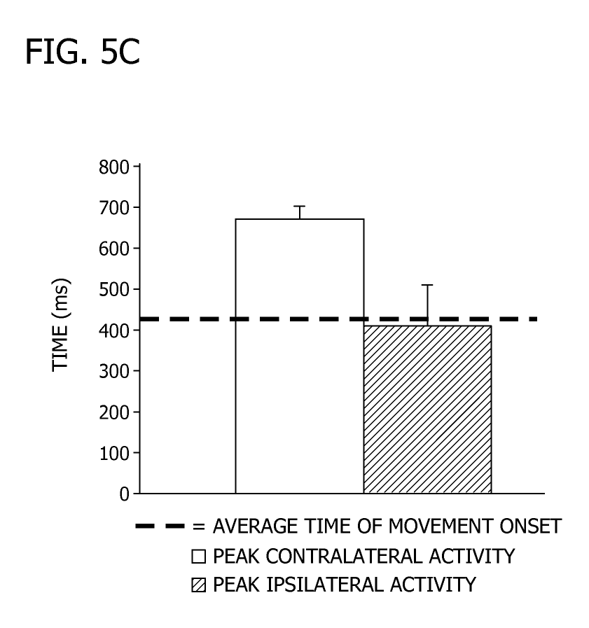

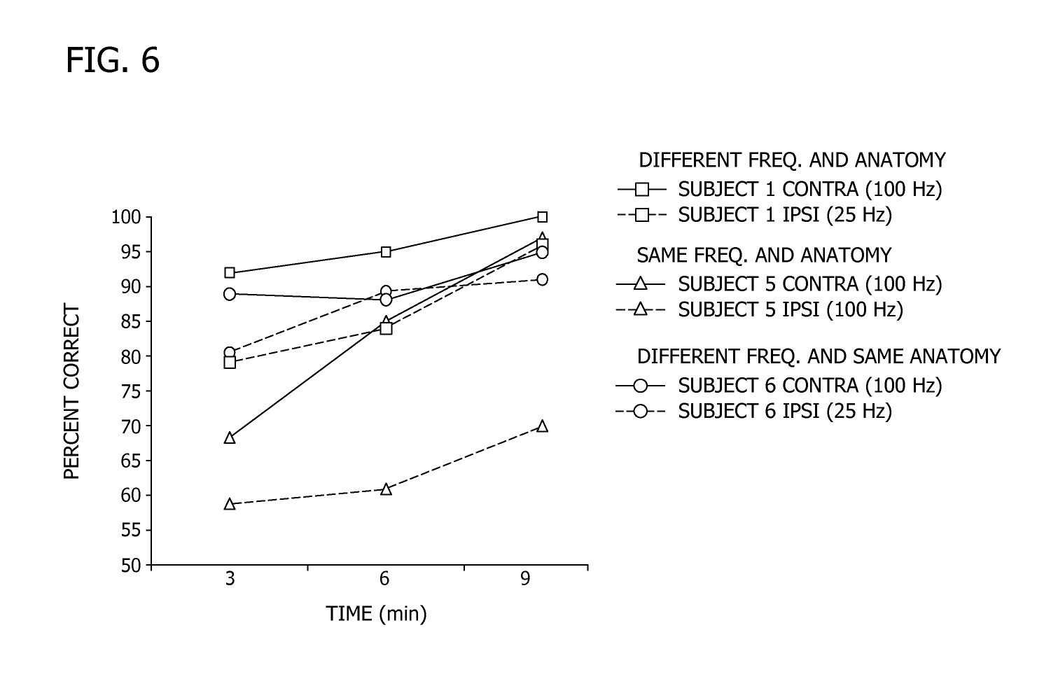

Pilot studies demonstrate the feasibility of the embodiments described herein. For example, to identify the separable cortical physiology associated with ipsilateral motor movements to supplement sensorimotor rhythm-derived BCI control, for gross hand movements, there are distinct anatomic sites and different timescales of cortical activity that are more highly represented in the lower frequencies than activity associated with contralateral movements, as shown in FIGS. 5A-5C. The earlier timescales and higher premotor localization support a motor planning role for ipsilateral motor processing. Additionally, these separable sites and spectra can be used for continuous cursor control, as shown in FIG. 6. These findings support the notion that ipsilateral motor physiology is distinct and can be used for adjunct BCI operation.

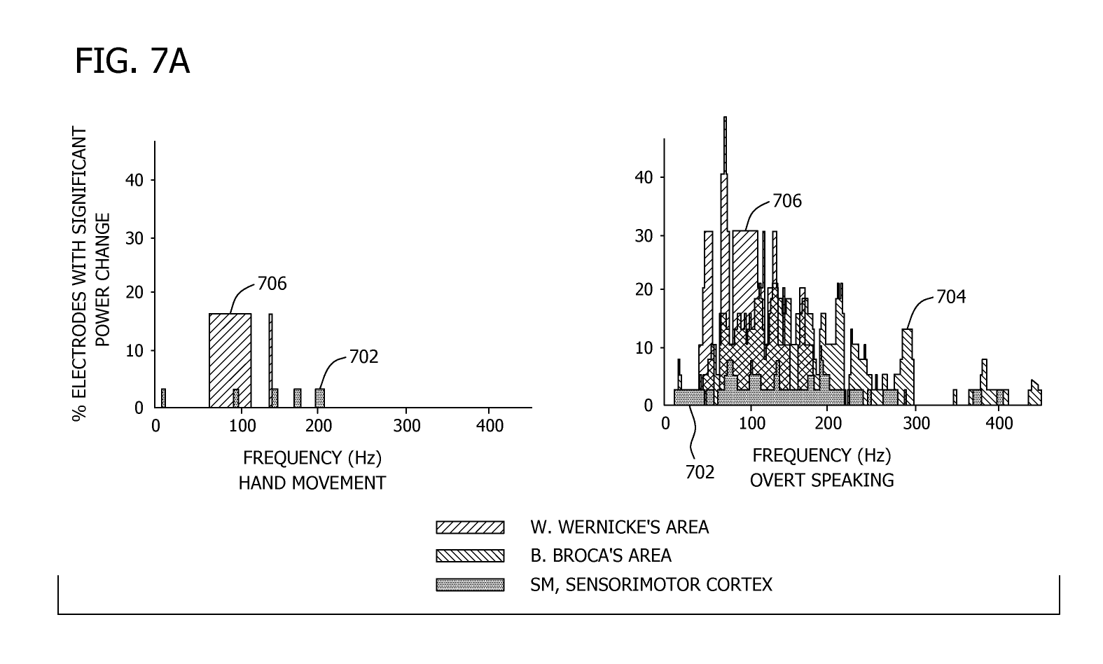

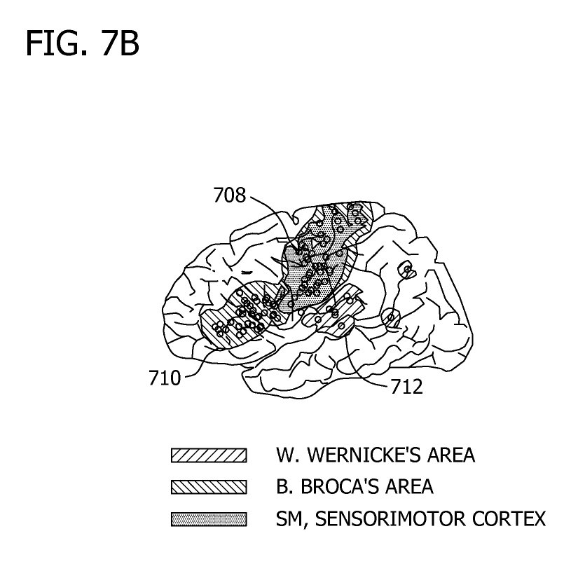

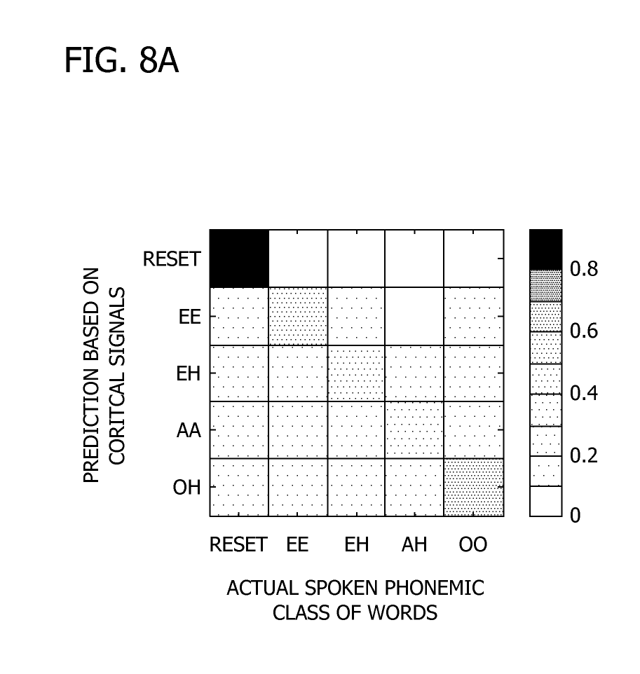

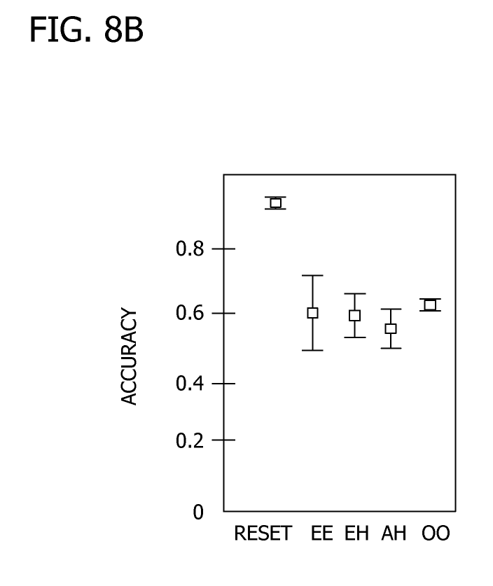

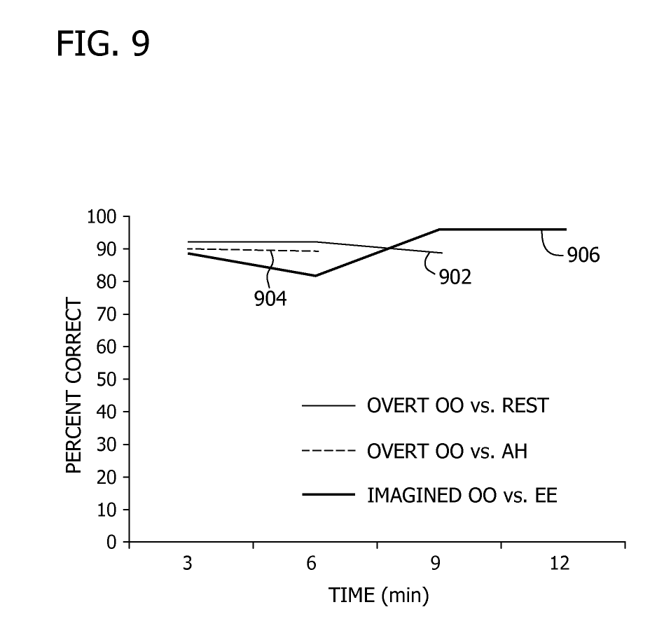

To identify the separable cortical physiology associated with speech to supplement sensorimotor rhythm derived BCI control, the embodiments described herein demonstrate that for human subjects performing both hand and speech tasks, there are very distinct cortical and spectral changes that distinguish the two cognitive intentions, as shown in FIG. 7. When further examined, four different phonemic classes of words can be differentiated using cortical and spectral ensembles, as shown in FIG. 8. FIG. 9 illustrates that these separable gamma features can be used for differential BCI operation. Embodiments of the disclosure contemplate that the wholly non-motor class of cognitive operations and physiology can constitute a potential set of control features for BCI.

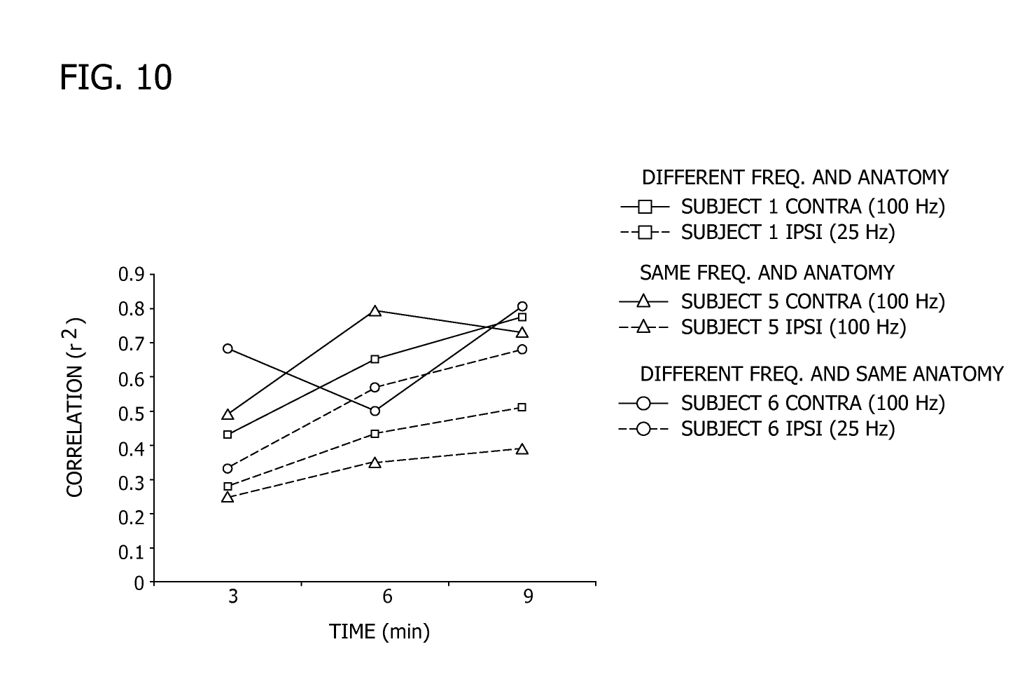

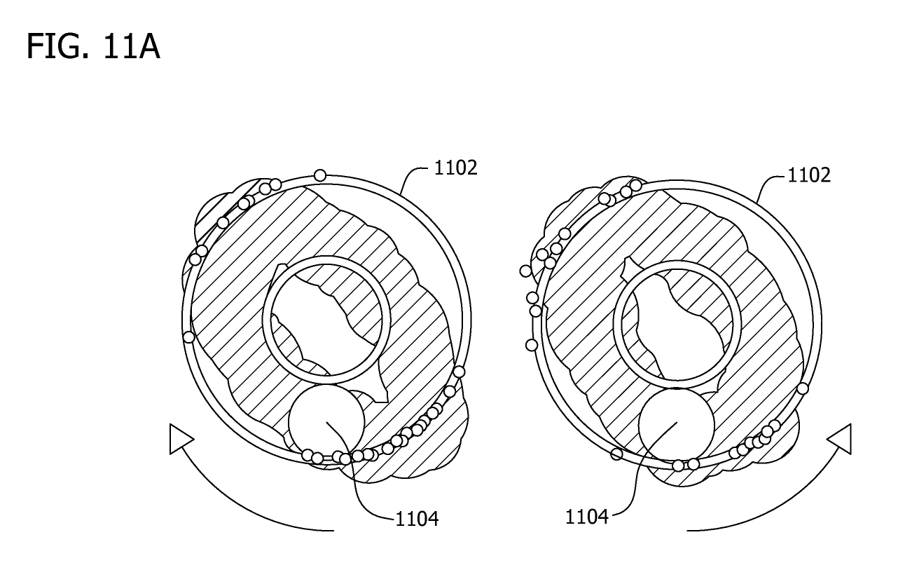

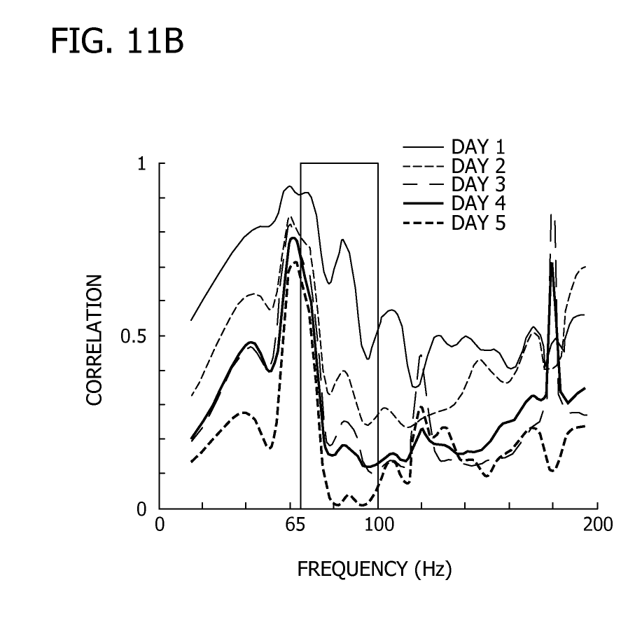

To define the role of biofeedback alone to enable creation of separable features to supplement sensorimotor rhythm-derived BCI control, the embodiments described herein illustrate that feedback induces changes in cortical features in human subjects in features previously identified in open-loop screening, as shown in FIG. 10. In a primate model, and as shown in FIG. 11, biofeedback alone is sufficient to create two independent control features without the need for prior screening. This finding has significant implications because it demonstrates that any region of the brain may have the potential to provide BCI control capability with appropriate biofeedback.

A study has been performed of thirteen human patients (ages 11-50 years) with intractable epilepsy who underwent temporary placement of intracranial electrode arrays to localize seizure foci prior to surgical resection. The subjects included six males, and seven females. All had normal levels of cognitive function and all were right-handed. In all experiments, ECoG signals were recorded from up to 64 electrodes using a general-purpose BCI system, such as BCl2000. All electrodes were referenced to an inactive intracranial electrode, amplified, bandpass-filtered (0.15-500 Hz), digitized at 1200 Hz, and stored. The amount of data obtained varied from patient to patient and depended on the patient's physical state and willingness to continue. The patients performed various hand, joystick, and language tasks (described below). The time series ECoG data was converted into the frequency domain. Various methods of signal analysis were performed to identify the most significant task-related physiologic changes. In a subset of six patients, closed-loop BCI experiments were attempted with the patient receiving online feedback that consisted of one-dimensional horizontal cursor movement controlled by ECoG features that had shown correlation with tasks during the screening procedures.

Additional preliminary data was acquired by invasively recording data from two monkeys. Epidural ECoG recordings were performed on two subjects that were prompted to control a computer cursor in two dimensions for a circle-drawing task. Arbitrarily identified cortical sites were used and horizontal and vertical velocities were pre-assigned to a gamma frequency band for each site. The subjects were given biofeedback alone to modulate the high gamma band power of the respective electrodes to control the horizontal and vertical velocity of the cursor.

The study showed that ipsilateral hand movement and movement imagery produce spectral changes located predominantly in premotor cortex and are associated with lower frequencies when compared to M1-sensorimotor rhythms associated with contralateral movements. These unique anatomic/spectral features provide an additional continuous control signal that can be used in parallel with control from M1-derived signals. Six subjects (1-6) performed an ipsilateral and contralateral hand motor task. This consisted of the subject performing repetitive three-second hand tasks consisting of opening and closing the right or left hand on cue (interspersed by a rest period of equal time). To precisely determine the timing of cue presentation, motor movement, and associated spectra changes, a subset of three subjects (1, 3, and 6) performed cue-directed hand-controlled joystick center-out tasks with both the right and left hand. The time-series ECoG data was converted into the frequency domain and each hand action was compared against rest. All subjects showed distinct frequency spectra and electrode sites that distinguished between the ipsilateral and contralateral hand movements.

FIG. 5A demonstrates two bar histograms in which the number of electrodes demonstrating significant cortical activity (spectral power changes with p-value <0.001) are plotted against frequency for ipsilateral and contralateral hand movements. Ipsilateral hand movements are predominantly represented by lower frequencies (average 32.8 Hz, SD+/−14.4) compared to the higher-frequency distribution associated with contralateral hand movements (average 106.7 Hz, SD+/−20.8). More specifically, the data shows the number of electrodes that exhibited significant power change (p-value<0.001) at a given frequency for ipsilateral and contralateral hand movements across six patients with intracranial grid arrays. Ipsilateral hand movements are represented in a lower frequency range than those associated with contralateral hand movements.

FIG. 5B compares the number of anatomic locations that showed significant changes in activity (electrodes showing spectral power changes with p-values <0.001) for ipsilateral and contralateral hand movements. The pie chart shows there are an equal number of cortical locations that are distinct to ipsilateral and contralateral hand movements. The adjacent bar histograms detail the relative representation of activation over sensorimotor cortex, premotor cortex, and non-motor cortex. These results demonstrate that ipsilateral movements are more highly represented within premotor cortex. The data demonstrates the number of locations identified with statistically significant power change (across all frequencies) that correlated with ipsilateral and contralateral hand movements. The number of sites that showed significant activity with ipsilateral hand movements are denoted in a first portion 502, contralateral hand movements are shown in a second portion 504, and locations that were shared by both ipsilateral and contralateral hand movements are shown in a third portion 506. This data showed that there are sites for ipsilateral motor movements that are distinct from contralateral hand movements. The anatomic distribution of ipsilateral and contralateral movement activations were also distinct with regard to Brodmann areas. Ipsilateral activations showed a larger representation in premotor regions as compared to contralateral activations, where S is sensorimotor signals detected in Brodmann areas one through 4, P is pre-motor signals detected in Brodmann area six, and N is non-motor signals detected in all other Brodmann areas. These findings support the idea that ipsilateral motor processing is more likely involved with motor planning than execution.

Finally, FIG. 5C presents a bar histogram that shows the peak time of signal correlation with the active condition (time of cue presentation/movement against rest) averaged across the three subjects performing the joystick task. Ipsilateral movements preceded similar changes with contralateral movements on average by 160 milliseconds (ms). The peak of signal correlation with the movement of a hand-operated joystick was averaged for three subjects (1, 5, and 6). Ipsilateral hand movement preceded contralateral hand movement by 160 ms. The dotted line is the initiation of movement. This again supports the idea that ipsilateral cortex is involved in a motor planning role in motor movements.

Collectively, FIGS. 5A-5C provide a key part of the basis for confirming that there are separable frequency spectra, cortical sites, and time scales that distinguish ipsilateral hand movements from contralateral hand movements. The cortical physiology and anatomic locations associated with ipsilateral movements is distinct and is involved in a more motor planning role that is separable from the cortical processing of contralateral motor execution within M1.

To determine whether signals associated with ipsilateral hand movements could be utilized, three of the six subjects (1, 5, and 6) who performed hand screening tasks (as described above) were also tested in a real-time online task to use features associated with either ipsilateral or contralateral overt hand movements to control a cursor on a computer screen. The patients received online feedback that consisted of one-dimensional horizontal cursor movement controlled by ECoG features that had showed correlation with either the ipsilateral or contralateral hand movements during open-loop screening. The goal of the task was to hit one of two specified targets. Each subject achieved closed-loop control twice, once using a contralateral hand task and a second time using an ipsilateral hand task. FIG. 6 illustrates that signals derived from ipsilateral motor movements can achieve high levels of control with final target accuracies between 70-96%. This control can be optimized when distinct locations and low-frequency spectra associated with ipsilateral movements are utilized. More specifically, FIG. 6 demonstrates the ability of the three subjects to utilize signals from a single hemisphere associated with either ipsilateral or contralateral hand movements to control a cursor on a computer screen. Each subject was different in what features were chosen to utilize for control. For example, subject 1 used different locations and different frequency spectra (ipsi—25 Hz, contra—100 Hz), subject 5 used identical locations and spectra (both ipsi and contra—100 Hz), and subject 6 used identical locations with different frequency spectra (ipsi—20 Hz, contra—100 Hz). These results demonstrate that optimal control can be achieved using either distinct locations or distinct frequency spectra. Performance when high frequency is utilized with ipsilateral hand movements is not as robust (dotted yellow line). FIG. 6 further illustrates that ECoG signals derived from ipsilateral hand movements can be utilized for device control, and that ipsilateral control signals can be differentiated from contralateral-derived control features both in cortical location and frequency spectra.

The study also showed that articulation of different phonemes produces distinct spectral changes in Broca's and Wernicke's areas. These unique anatomic/spectral features provide additional discrete control signals that can be used in parallel with continuous control from M1-derived signals as a discrete selector (e.g., a computer mouse “click” function). Seven subjects (7-13) with electrode arrays covering putative speech areas performed a language task while being invasively monitored. During open-loop recording, subjects verbally repeated stimulus words they heard through ear phones. Each stimulus word consisted of a consonant-vowel-consonant (CVC) combination. Each set of nine words had one out of four vowels, the set of which collectively spanned the vowel articulation space: [ee], [eh], [oo] and [ah] as in “read,” “red,” “rude” and “rad.” Thus, a total of 36 unique words were presented. Prior to beginning the experiment, subjects were individually informed of the experimental paradigm and instructed to speak the word that they heard. The 36 CVC word set were presented individually and pseudo-randomly. The set was repeated as many times as the subject's condition and willingness to proceed permitted. Additionally, a subset of five patients also had hand motor coverage and performed motor tasks consisting of cue-initiated right hand opening and closing.

FIGS. 7A and 7B illustrate that there are different anatomic and spectral characteristics unique to speech articulation that are not present with hand movements. Anatomically, this can be seen specifically with frequency alterations (as shown in FIG. 7A) in Broca's area (shown in FIG. 7B) that are present only with overt speech (and not present with hand movements). Spectrally there is also a notable higher frequency content in all regions with speech when compared to hand movement. Specifically, overt speech induces power changes above 200 Hz in sensorimotor cortex that does not occur with hand movement. Additionally, overt speech has higher spectral changes between 150-200 Hz in Wernicke's area that are also not present with hand movements.

In FIG. 7A, the data shows the percentage of electrodes to a given region that exhibited significant power change (p-value<0.001) at a given frequency for overt hand movements and for overt speech articulation. The three regions evaluated were sensorimotor cortex 702, Broca's area 704, and Wernicke's area 706. The data shows that there are separable frequency bands that distinguish speech and motor intentions. Most notably, Broca's area 704 demonstrates a broad frequency change for which there is no associated change at all with hand movements. The sensorimotor cortex shows a higher frequency content above 200 Hz with speech that is not present with hand movements. Wernicke's area 706 also shows a higher frequency content above 150 Hz that is not seen with hand movements. FIG. 7B illustrates the anatomic location of the electrodes (summated across patients) that were recorded from during motor and speech tasks. The electrodes were grouped according to three functional regions: 1) sensorimotor cortex (SM) 708; 2) Broca's area (B) 710; and 3) Wernicke's area (W) 712. This demonstrates that there are distinct anatomic regions and frequency bands that can be used to distinguish a motor intention from a speech intention.

The data was further taken from five of the seven previous subjects and further evaluated to define whether anatomic and spectral ensembles could provide additional information and further discriminate phonemic content. In order to assess the information present in neural ensembles, or networks, a principal component analysis (PCA) was performed on cortical signals from all electrodes for a given subject prior to conditional comparisons. This defined the degree to which certain cortical sites co-varied in time together. Not all regions showing covariance, however, have functional or informational significance. Thus, a Discriminant Function Analysis (DFA) was utilized to explicitly test which co-varying patterns maximally correlated to an event in time, namely the articulation of different phonemic classes of words. As shown in FIGS. 8A and 8B, cortical ensembles carry substantial information in classifying different phonemic classes of words. FIG. 8A shows that the average classification accuracy of each of the four phonemic classes was 58%. Moreover, as shown in FIG. 8A, the data shows the percentage of recorded ECoG sample points correctly classified as “Rest,” “EE,” “EH,” “AH,” and “OO” using network patterns defined by functionally connected cortical sites. FIG. 8B illustrates that the mean and standard error range across the five subjects was substantially above chance (20%) for each of the four phonemic classes and rest. Moreover, FIG. 8B illustrates the mean accuracy across subjects with standard error. The chance accuracy for the five conditions is 20% (indicated by the dotted line). These findings show that there is substantial information within ECoG signals sufficient to distinguish different phonemic articulations.

To determine whether signals associated with the different phonemic classes of speech articulation could be used for device control, three of the seven subjects who performed the speech screening tasks (as described above) also were tested in a real-time online task using features associated with phonemes to make a two-choice selection on a computer screen. The patients received online feedback that consisted of selecting one of two targets on a screen using ECoG features that had showed correlation with a given phoneme articulation (either overt or imagined). The goal of the task was to select one of two specified targets. Three scenarios were tested: overt phoneme vs. rest (line 902 in FIG. 9), overt phoneme vs. phoneme (line 904), and imagined phoneme vs. phoneme (line 906). Each subject achieved a high level of performance quickly. FIG. 9 illustrates that signals derived from speech can be used for very effective choice selection. High levels of control were achieved immediately with little or no learning curves. Initial accuracies for the first three minutes were between 88% and 92%; final accuracies were between 88% and 96%. Moreover, FIG. 9 demonstrates the ability of three subjects to utilize signals from frontal speech sites associated with either overt or imagined phoneme articulation to select a target on a computer screen. The patients received online feedback that consisted of selecting one of two targets on a screen using ECoG features that had showed correlation with a given phoneme articulation (either overt or imagined). These results demonstrate that signals derived from speech intentions provide a strong substrate for BCI operation with little training while enabling subjects to achieve high performance quickly. Moreover, these results show that the separable characteristics associated with phoneme articulation (both real and imagined) can indeed be utilized for BCI control that may provide additional control features when used in conjunction with sensorimotor rhythms.

With appropriate feedback, subjects can alter the amplitude of designated frequencies at designated locations to create “de novo” control features which provide additional discrete control signals that can be used in parallel with continuous control from M1-derived signals as a discrete selector (e.g., a computer mouse “click” function). As described above, three subjects used cortical features associated with previously screened contralateral and ipsilateral hand movements to control the horizontal movement of a cursor on a screen. In addition to the control performance shown in FIG. 10, the control features themselves were analyzed to define their change with ongoing performance. The time-series ECoG data during the control session was converted into the frequency domain and each control feature was compared against rest for each 3-minute run that the patient participated in. The correlation of amplitude change for the specified frequency band was defined relative to the correct target. As shown in FIG. 10, all features showed increasing correlation (as measured by the cross-correlation coefficient, r2) to the desired target over time. This was true for both higher (100 Hz) and lower (25 Hz) frequencies. Higher frequencies, however, appeared to show higher correlations with ongoing performance than the lower frequencies. For example, FIG. 10 illustrates the level of correlation (as measured by r2) with the selected frequency bands utilized for control with the respective targets. Over time, all signals showed increased correlation, demonstrating that these signals exhibit dynamic changes with ongoing feedback. These results support the notion that feedback (in the form of cursor movement) has an impact on altering human cortical physiology over time. These feedback-induced changes occur quickly and more robustly at higher gamma frequencies.

As described above, epidural ECoG recordings were performed in two monkeys who were trained to control a computer cursor in two dimensions for a circle-drawing task, as shown in FIGS. 11A and 11B. By randomly choosing two epidural cortical locations spaced one centimeter apart, the monkeys were able to modulate the high gamma band power under these two electrodes to control the horizontal and vertical velocity of the cursor. Using solely biofeedback (e.g., no adaptation in the decoding algorithm), the monkeys were able to train the population of neurons under each 380-micron micro-ECoG electrode to accurately modulate their high gamma band activity (65-100 Hz) to control the 2D cursor. As shown in FIG. 11B, since the electrodes were a centimeter apart, the signals were initially quite correlated on the first day. After a week of training, however, the monkeys learned to independently control the gamma band activity under each electrode such that their activity completely de-correlated in the circle-drawing task. These findings support the nascent plastic ability of the brain to alter its own activity based on feedback.

In FIG. 11A, lines 1102 indicate the average cursor position during the third day of the closed-loop circle task for clockwise circles (left) and counterclockwise circles (right). Circles 1104 indicate the points at which the cursor left the circle. As shown in FIG. 11B, to achieve two-dimensional control, the amplitude of the signal between 65-100 Hz from one epidural ECoG electrode was used as the control for the horizontal velocity of the cursor and a separate electrode was used for the vertical velocity of the cursor. To achieve full control in a circle-drawing task, it was necessary for the animal to gain independent control of the two electrode signals. For a perfectly drawn circle, the overall correlation between the two signals is zero. The figure shows the resulting correlations for the five days of recordings. This shows that the correlation between the recording sites dropped across most frequencies but most dramatically between 65-100 Hz. Therefore, this data clearly shows that through biofeedback, the cortex is quite adaptable to learning and improving brain-computer interface control without prerequisite screening. These findings show that biofeedback alone (without screening) is sufficient to enable a primate brain to alter its cortical signal sufficiently to achieve a high level of neuroprosthetic performance. This ability to alter a signal provides the potential opportunity to create de novo features to complement sensorimotor signal-derived control in humans.

The embodiments described herein illustrate that 1) ipsilateral hand movements have a distinct cortical physiology that can be used for BCI operation; 2) there are separable anatomic and spectral features associated with the articulation of speech that are distinct from motor intentions; 3) different components of speech articulation (phonemes) can be distinguished using ECoG and that the features associated with individual phonemes can be used for BCI operation; 4) control signals show a high level of plasticity that change quickly with feedback in humans; and 5) feedback alone is sufficient to produce separable control signals if subjects are given enough time and feedback in primate models.

Exemplary embodiments of systems, methods, and apparatus for determining a cognitive task based on one more cortico-physiologies are described above in detail. The systems, methods, and apparatus are not limited to the specific embodiments described herein but, rather, operations of the methods and/or components of the system and/or apparatus may be utilized independently and separately from other operations and/or components described herein. Further, the described operations and/or components may also be defined in, or used in combination with, other systems, methods, and/or apparatus, and are not limited to practice with only the systems, methods, and storage media as described herein.

A computer, such as that described herein, includes at least one processor or processing unit and a system memory. The computer typically has at least some form of computer readable media. By way of example and not limitation, computer readable media include computer storage media and communication media. Computer storage media include volatile and nonvolatile, removable and non-removable media implemented in any method or technology for storage of information such as computer readable instructions, data structures, program modules, or other data. Communication media typically embody computer readable instructions, data structures, program modules, or other data in a modulated data signal such as a carrier wave or other transport mechanism and include any information delivery media. Those skilled in the art are familiar with the modulated data signal, which has one or more of its characteristics set or changed in such a manner as to encode information in the signal. Combinations of any of the above are also included within the scope of computer readable media.

Although the present invention is described in connection with an exemplary computer system environment, embodiments of the invention are operational with numerous other general purpose or special purpose computer system environments or configurations. The computer system environment is not intended to suggest any limitation as to the scope of use or functionality of any aspect of the invention. Moreover, the computer system environment should not be interpreted as having any dependency or requirement relating to any one or combination of components illustrated in the exemplary operating environment. Examples of well known computer systems, environments, and/or configurations that may be suitable for use with aspects of the invention include, but are not limited to, personal computers, server computers, hand-held or laptop devices, multiprocessor systems, microprocessor-based systems, set top boxes, programmable consumer electronics, mobile telephones, network PCs, minicomputers, mainframe computers, distributed computing environments that include any of the above systems or devices, and the like.

Embodiments of the invention may be described in the general context of computer-executable instructions, such as program components or modules, executed by one or more computers or other devices. Aspects of the invention may be implemented with any number and organization of components or modules. For example, aspects of the invention are not limited to the specific computer-executable instructions or the specific components or modules illustrated in the figures and described herein. Alternative embodiments of the invention may include different computer-executable instructions or components having more or less functionality than illustrated and described herein.

The order of execution or performance of the operations in the embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

In some embodiments, the term “processor” refers generally to any programmable system including systems and microcontrollers, reduced instruction set circuits (RISC), application specific integrated circuits (ASIC), programmable logic circuits (PLC), and any other circuit or processor capable of executing the functions described herein. The above examples are exemplary only, and thus are not intended to limit in any way the definition and/or meaning of the term processor.

When introducing elements of aspects of the invention or embodiments thereof, the articles “a,” “an,” “the,” and “said” are intended to mean that there are one or more of the elements. The terms “comprising,” including,” and “having” are intended to be inclusive and mean that there may be additional elements other than the listed elements.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method comprising:
establishing communication with one or more electrodes for acquiring brain signals of a subject;
interrogating a brain of a subject concurrently via the one or more electrodes;
concurrently receiving signals representative of a plurality of cortico-physiologies within the subject's brain; and
transmitting signals representative of the plurality of cortico-physiologies to a computer for use in controlling operation of a device.

2. The method of claim 1, wherein interrogating the brain of the subject comprises acquiring the signals representative of the plurality of cortico-physiologies from multiple locations within the brain.

3. The method of claim 1, wherein interrogating the brain of the subject comprises acquiring the signals representative of the plurality of cortico-physiologies from a single location within the brain which represent different cognitive operations.

4. The method of claim 3, wherein the different cognitive operations include one or more of the following: motor processing, speech processing, attention, memory, visual processing, and auditory processing.

5. The method of claim 1, wherein interrogating the brain of the subject comprises acquiring one or more of:
primary motor signals from the brain;
non-primary motor signals from the brain;
motor signals from the brain; and
non-motor signals from the brain.

6. The method of claim 1, further comprising decoding the signals representative of the plurality of cortico-physiologies to determine an intended action by the subject and controlling the device based on the decoded signals.

7. The method of claim 1, wherein concurrently receiving signals representative of a plurality of cortico-physiologies within the subject's brain comprises one or more of:
receiving electrocorticograhy (ECoG) cortical signals;
receiving electroencephaplography (EEG) cortical signals;
receiving local field potential signals;
receiving single neuron signals;
receiving magnetoencephalography (MEG) signals; and
receiving multi-unit (mu) activity signals.

8. An apparatus comprising:
a memory area configured to store a correlation between cortico-physiologies and intended actions;
an interface configured to receive brain signals from a subject via one or more electrodes; and
a processor configured to:
detect, from the brain signals received from the interface, at least one of the cortico-physiologies; and
identify at least one of the intended actions correlating to the detected cortico-physiologies.

9. The apparatus of claim 8, wherein the interface is configured to receive the brain signals from multiple locations within the brain.

10. The apparatus of claim 8, wherein the interface is configured to receive the brain signals from a single location within the brain and distinguish at least two cortico-physiologies that represent at least two cognitive operations.

11. The apparatus of claim 8, wherein the brain signals include primary motor signals and non-primary motor signals.

12. The apparatus of claim 8, wherein the brain signals include motor signals and non-motor signals.

13. The apparatus of claim 8, wherein the processor is further configured to record the brain signals in a memory area and to decode the brain signals to generate a control signal.

14. One or more computer-readable storage media having computer-executable components, the components comprising:
a communication component that when executed by at least one processor causes the at least one processor to receive brain signals from a subject via one or more electrodes, wherein the brain signals are representative of a plurality of physiologies;
a signal analysis component that when executed by at least one processor causes the at least one processor to determine at least one cognitive task associated with the plurality of physiologies; and
a control component that when executed by at least one processor causes the at least one processor to perform an action related to the at least one cognitive task.

15. The computer-readable storage media of claim 14, wherein the signal analysis component causes the at least one processor to decode the brain signals to determine the at least one cognitive task.

16. The computer-readable storage media of claim 14, wherein the control component causes the at least one processor to control a device based on the action related to the at least one cognitive task.

17. The computer-readable storage media of claim 14, wherein the brain signals are acquired by the one or more electrodes from multiple locations within the subject's brain.

18. The computer-readable storage media of claim 14, wherein the brain signals are concurrently acquired by the one or more electrodes from multiple locations within the subject's brain.

19. The computer-readable storage media of claim 14, wherein the brain signals include primary motor signals and non-primary motor signals.

20. The computer-readable storage media of claim 14, wherein the brain signals include motor signals and non-motor signals.
